(19) United States
(12) Patent Application Publication
(10) Pub. No.: US 2017/0287353 A1
(43) Pub. Date: Oct. 5, 2017

(54) ALCOHOL AND DRUG INTERVENTION SYSTEM AND METHOD

(71) Applicants: Donna KAZEMI, Charlotte, NC (US); Mohamed SHEHAB, Charlotte, NC (US); Beau DOOLEY, Charlotte, NC (US)

(72) Inventors: Donna KAZEMI, Charlotte, NC (US); Mohamed SHEHAB, Charlotte, NC (US); Beau DOOLEY, Charlotte, NC (US)

(73) Assignee: UNIVERSITY OF NORTH CAROLINA AT CHARLOTTE, Charlotte, NC (US)

(21) Appl. No.: 15/476,023

(22) Filed: Mar. 31, 2017

Related U.S. Application Data
(60) Provisional application No. 62/317,138, filed on Apr. 1, 2016.

(51) Int. Cl. G09B 19/00; G09B 5/06; G09B 7/08; G01N 33/497; A61B 5/08; A61B 5/145; A61B 5/15; A61B 10/00; G09B 7/02; A61B 5/00

(52) U.S. Cl. CPC G09B 19/00; G09B 7/02; G09B 5/065; G09B 7/08; A61B 5/0077; A61B 5/082; A61B 5/14507; A61B 5/15; A61B 5/4845; A61B 5/6898; A61B 5/486; A61B 5/746; A61B 10/0051; G01N 33/497; H04L 67/42

(57) ABSTRACT

An alcohol and drug intervention system and method, comprising: a server component; and a mobile application component wirelessly coupled to the server component; wherein the server component and the mobile application component are collectively operable for, in an interactive manner, receiving input from a user and presenting the user with output based on the input received from the user, wherein the input received from the user comprises one or more of personal information, activity information, and activity related to alcohol and/or drug use, and wherein the output presented to the user comprises one or more of coaching information and counseling information. Optionally, the system further comprises a testing device coupled to the mobile application component and adjusting one or more of the coaching and counseling information based on results obtained from the testing device.

### CROSS-REFERENCE TO RELATED APPLICATION

The present patent application/patent claims the benefit of priority of co-pending U.S. Provisional Patent Application No. 62/317,138, filed on Apr. 1, 2016, and entitled “COMPUTER IMPLEMENTED DELIVERY OF A MOTIVATIONAL INTERVENTION TO ADDRESS HEAVY ALCOHOL CONSUMPTION AND OTHER DISORDERS,” the contents of which are incorporated in full by reference herein.

### STATEMENT OF GOVERNMENT SPONSORED RESEARCH AND/OR DEVELOPMENT

The present invention was made with Government support by the Agency for Healthcare Research and Quality (AHRQ), Award No. 1R21 HSO23875-01. Accordingly, the Government has certain rights in the present invention.

### FIELD OF THE INVENTION

The present invention relates generally to an alcohol and drug intervention system and method. More specifically, the present invention relates to an alcohol and drug intervention system and method that periodically queries and/or messages a user about their alcohol and drug use. This periodicity may be random, scheduled, or prompted by a geofencing application or the like. The intervention system includes a server component and a mobile application component executed on a wireless mobile device or the like. Optionally, the intervention system also includes a physical testing device, such as a breathalyzer, a fluid tester, or the like, that is coupled to the wireless mobile device. The intervention system utilizes robust data security technologies and implements novel archiving and, optionally, reporting features.

### BACKGROUND OF THE INVENTION

Alcohol and drug use among college students is a national public health concern, resulting in significant consequences among college students 18-24 years old. College administrators continually seek to reduce heavy drinking and drug use and mitigate these consequences, often at great effort and expense. Multiple intervention approaches are utilized, including the dissemination of public awareness information and online and in person coaching and therapy, the latter of which is typically confidential. Many of the packaged intervention tools are utilized at the over 4,000 public and private institutions of higher learning in the United States. These intervention tools are typically subscription based and the cost structure is often defined by student enrollment. For example, some of the intervention tools cost between $1,000 and $15,000 per year, creating a market of approximately $60 million per year.

Because today's students are digital natives and mobile devices are ubiquitous among them, a mobile application designed to moderate alcohol and drug use could greatly help and overcome logistical and financial barriers in higher education.

### BRIEF SUMMARY OF THE INVENTION

Thus, in various exemplary embodiments, the present invention provides a mobile application that motivates college students (and potentially others) to reduce excessive drinking and drug use, and the associated harms, by translating face-to-face brief motivational interventions (BMIs) into a digital format utilizing a virtual coach, motivational interviewing, personalized feedback, educational games, and user tools (e.g. goal setting features, harm reduction strategies, drink/drug logs, resources, etc.). Based on an inputted user profile, the intervention system periodically queries and/or messages a user about their alcohol and drug use. This periodicity may be random, scheduled, or prompted by a geofencing application or the like. The intervention system includes a server component and a mobile application component executed on a wireless mobile device or the like. Optionally, the intervention system also includes a physical testing device, such as a breathalyzer, a fluid tester, or the like, that is coupled to the wireless mobile device. The intervention system utilizes robust data security technologies and implements novel archiving and, optionally, reporting features. As a whole, the intervention system periodically assess the user's status in terms of alcohol or drug use and provides appropriate coaching. The coaching is based on motivational interviewing and incorporates both user persuasion and health research. Informational and interventional output, trivia, games, a blood alcohol content (BAC) calculator, and a log book are all provided, for example, and user feedback allows for a great deal of personalization.

In one exemplary embodiment, the present invention provides an alcohol and drug intervention system, comprising: a server component; and a mobile application component wirelessly coupled to the server component; wherein the server component and the mobile application component are collectively operable for, in an interactive manner, receiving input from a user and presenting the user with output based on the input received from the user, wherein the input received from the user comprises one or more of personal information, activity information, and activity related to alcohol and/or drug use, and wherein the output presented to the user comprises one or more of coaching information and counseling information. The server component comprises a processor executing one or more application programming interfaces and a database. The mobile application component comprises one or more of a mobile telephone, a personal digital assistant device, and a tablet device. The server component and the mobile application component are further collectively operable for presenting trivia game questions to the user and receiving trivia game answers from the user and adjusting one or more of the coaching and counseling information based on the trivia game answers received from the user. The server component and the mobile application component are further collectively operable for implementing a blood alcohol content calculator. Optionally, the system further comprises a geofencing component operable for determining a location of the user and tailoring requested inputs and presented outputs based thereon. Optionally, the system still further comprises an alarm component operable for one or more of triggering and alarm and alerting a third party if the input received from the user indicates that user alcohol and/or drug use has exceeded a predetermined threshold. Optionally, the system still further comprises a testing device coupled to the mobile application component and adjusting one or more of the coaching and counseling information based on results obtained from the testing device. The testing device comprises one or more of a breathalyzer, a saliva testing device, a blood testing device, a urine testing device, a facial expression analyzer. Optionally, the server component and the mobile application component are further collectively operable for blinding an identity of the user such that the user is anonymous when interacting with the mobile application component.

In another exemplary embodiment, the present invention provides an alcohol and drug intervention method, comprising: providing a server component; and providing a mobile application component wirelessly coupled to the server component; wherein the server component and the mobile application component are collectively operable for, in an interactive manner, receiving input from a user and presenting the user with output based on the input received from the user, wherein the input received from the user comprises one or more of personal information, activity information, and activity related to alcohol and/or drug use, and wherein the output presented to the user comprises one or more of coaching information and counseling information. The server component comprises a processor executing one or more application programming interfaces and a database. The mobile application component comprises one or more of a mobile telephone, a personal digital assistant device, and a tablet device. The server component and the mobile application component are further collectively operable for presenting trivia game questions to the user and receiving trivia game answers from the user and adjusting one or more of the coaching and counseling information based on the trivia game answers received from the user. The server component and the mobile application component are further collectively operable for implementing a blood alcohol content calculator. Optionally, the method further comprises providing a geofencing component operable for determining a location of the user and tailoring requested inputs and presented outputs based thereon. Optionally, the method still further comprises providing an alarm component operable for one or more of triggering and alarm and alerting a third party if the input received from the user indicates that user alcohol and/or drug use has exceeded a predetermined threshold. Optionally, the method still further comprises providing a testing device coupled to the mobile application component and adjusting one or more of the coaching and counseling information based on results obtained from the testing device. The testing device comprises one or more of a breathalyzer, a saliva testing device, a blood testing device, a urine testing device, a facial expression analyzer. Optionally, the server component and the mobile application component are further collectively operable for blinding an identity of the user such that the user is anonymous when interacting with the mobile application component.

### BRIEF DESCRIPTION OF THE DRAWINGS

The present invention is illustrated and described herein with reference to the various drawings, in which like reference numbers are used to denote like system components/method steps, as appropriate, and in which:

FIG. 1 is a mobile application screenshot of an exemplary coaching introduction and query screen associated with the intervention system of the present invention;

FIG. 2 is a mobile application screenshot of an exemplary coaching output screen associated with the intervention system of the present invention, distilling collected and archived information into statistical conclusions;

FIG. 3 is a mobile application screenshot of an exemplary trivia game screen associated with the intervention system of the present invention;

FIG. 4 is a schematic diagram illustrating one exemplary embodiment of the structural architecture of the intervention system of the present invention, including a server component, a mobile application component, and, optionally, a pluggable testing device;

FIG. 5 is a mobile application screenshot of an exemplary authentication screen associated with the intervention system of the present invention;

FIG. 6 is a mobile application screenshot of an exemplary coaching output screen associated with the intervention system of the present invention;

FIG. 7 is a mobile application dual screenshot of an exemplary coaching output screen associated with the intervention system of the present invention;

FIG. 8 is a mobile application screenshot of an exemplary coaching output screen associated with the intervention system of the present invention;

FIG. 9 is a mobile application screenshot of an exemplary BAC calculator screen associated with the intervention system of the present invention;

FIG. 10 is a mobile application screenshot of an exemplary learning resource screen associated with the intervention system of the present invention;

FIG. 11 is a mobile application screenshot of an exemplary contact list screen associated with the intervention system of the present invention;

FIG. 12 is a mobile application screenshot of an exemplary trivia game screen associated with the intervention system of the present invention;

FIG. 13 is a mobile application screenshot of an exemplary trivia game scoreboard screen associated with the intervention system of the present invention; and

FIG. 14 is a mobile application screenshot of an exemplary trivia game feedback screen associated with the intervention system of the present invention.

### DETAILED DESCRIPTION OF THE INVENTION

Again, in various exemplary embodiments, the present invention provides a mobile application that motivates college students (and potentially others) to reduce excessive drinking and drug use, and the associated harms, by translating face-to-face BMIs into a digital format utilizing a virtual coach, motivational interviewing, personalized feedback, educational games, and user tools (e.g. goal setting features, harm reduction strategies, drink/drug logs, resources, etc.). Based on an inputted user profile, the intervention system periodically queries and/or messages a user about their alcohol and drug use. This periodicity may be random, scheduled, or prompted by a geofencing application or the like. The intervention system includes a server component and a mobile application component executed on a wireless mobile device or the like. Optionally, the intervention system also includes a physical testing device, such as a breathalyzer, a fluid tester, or the like, that is coupled to the wireless mobile device. The intervention system utilizes robust data security technologies and implements novel archiving and, optionally, reporting features. As a whole, the intervention system periodically assess the user's status in terms of alcohol or drug use and provides appropriate coaching. The coaching is based on motivational interviewing and incorporates both user persuasion and health research. Informational and interventional output, trivia, games, a BAC calculator, and a log book are all provided, for example, and user feedback allows for a great deal of personalization.

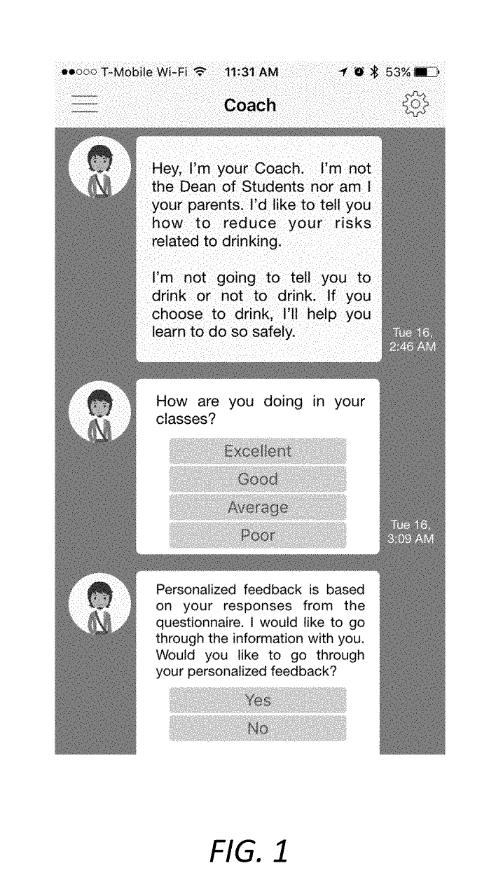

FIG. 1 is a mobile application screenshot of an exemplary coaching introduction and query screen associated with the intervention system of the present invention.

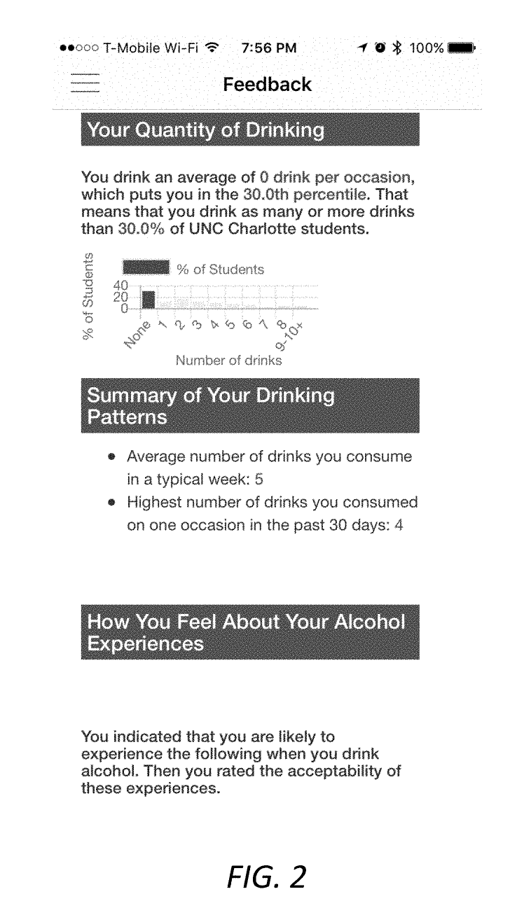

FIG. 2 is a mobile application screenshot of an exemplary coaching output screen associated with the intervention system of the present invention, distilling collected and archived information into statistical conclusions.

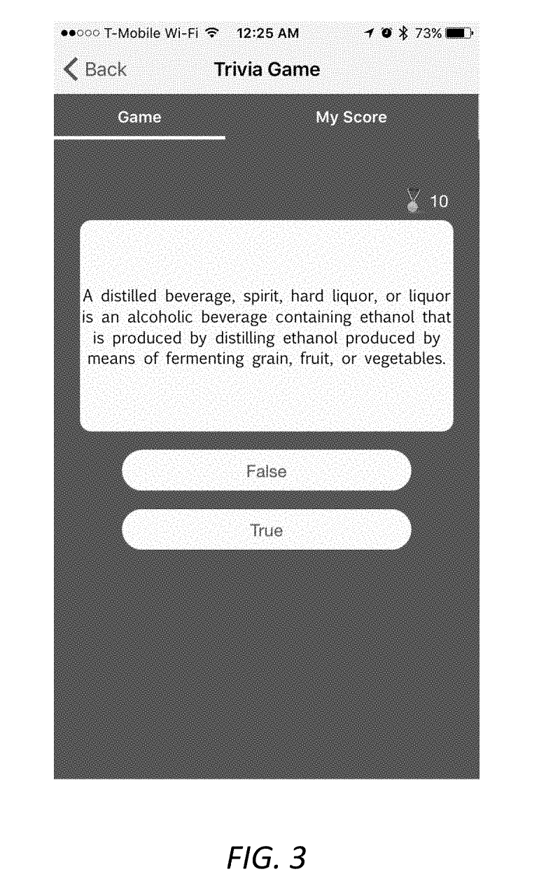

FIG. 3 is a mobile application screenshot of an exemplary trivia game screen associated with the intervention system of the present invention.

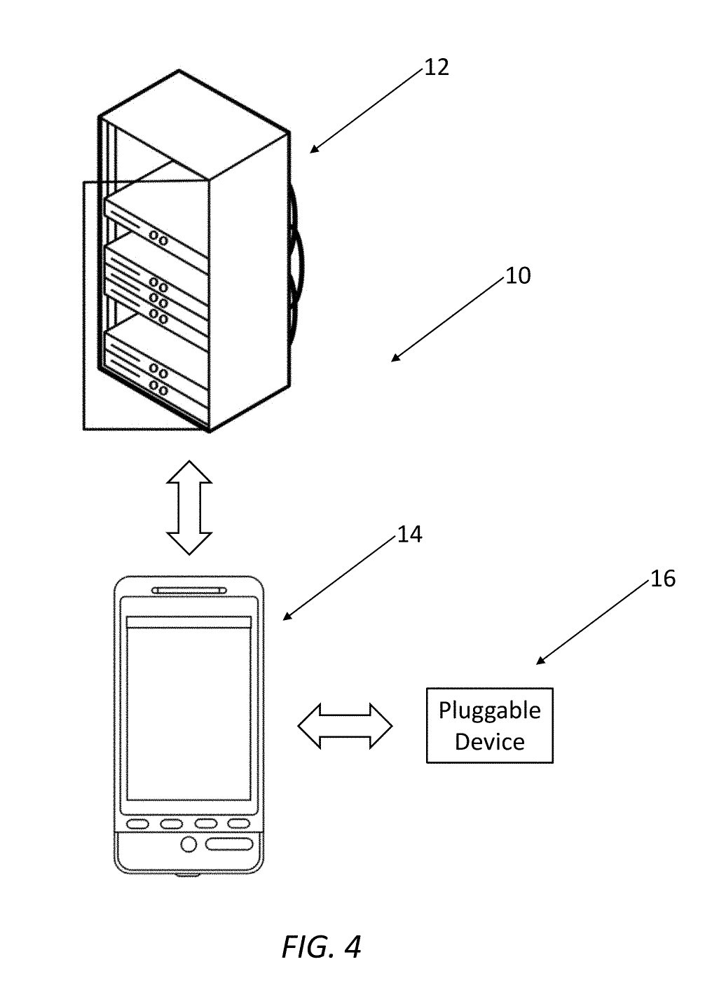

FIG. 4 is a schematic diagram illustrating one exemplary embodiment of the structural architecture of the intervention system 10 of the present invention, including a server component 12, a mobile application component 14, and, optionally, a pluggable testing device 16.

The server component 12 is designed and hosted on a managed network server that resides in a data center or the like. The server uses a Java and Spring architecture, for example. The server provides storage for all mobile application data, all survey and web functions, and application programming interfaces (APIs) required to interface with the mobile application. All user and other data is stored on the server in a MySQL database or the like. Interfacing with the database is performed using the Hibernate framework or the like. Stored data items include, but are not limited to, user profile information, user survey responses, user customized feedback, user strategies, trivia and game categories/questions/scoreboards, user customized conversations, user responses generated through the mobile application, other statistics collected through the mobile application, etc. Services provided, but are not limited to, user surveys, user feedback, push notifications, background job management, storage services, APIs, etc. User surveys include, but are not limited to demographic surveys, AUDIT surveys, DDQ surveys, B-YAACQ surveys, etc. The user feedback function provides customized feedback to the user based on information provided in the user surveys. The background job function is a background job on the server that executes periodically (every thirty minutes, for example), performing queries on the databases to identify users to contact and to send push notifications to. The push notifications function sends push notifications to users to alert the users to different components and to ask users specific questions based on the customized conversation. Finally, the APIs include user authentication (login, logout, etc.), user profiles (get user profile, update user profile, etc.), coaching (get coaching list, set answer to coaching message, update coaching message open list, etc.), user feedback (get user feedback, etc.), trivia (get trivia questions, set user trivia answers, get trivia scoreboard list, etc.), winning rounds (set user winning rounds time, get winning rounds scoreboard list, etc.), push notifications (register user device token, unregister user device token, etc.), etc.

Of note, the optional geofencing functionality allows the mobile application to use GPS or near field location information to identify where a user is at a given time and, if that location is determined to be an “at risk” location (based on publicly available information or a prior tag), such as a bar or a known acquaintance's residence, provide the user with immediate event prompted coaching. This event prompted coaching may be preceded by a user alert or the like.

The mobile application component 14 is developed for and iOS or Android framework, for example, using Swift or the like. The mobile application communicates with the server using the developed API services and through push notifications. The mobile application maintains a local database (Core Data) that is used to store, user profiles, user session information, and coaching interactions with users.

Further, the optional pluggable testing device 16 can include a breathalyzer, a saliva testing device, a blood testing device, a urine testing device, a facial expression analyzer, or the like that is coupled to the data port or the headphone jack of the mobile device. The user may be periodically prompted to use this pluggable testing device 16, such as at regular intervals, responsive to a given questionnaire, at the direction of the geofencing application, etc., and, depending upon the testing results, the user is then provided with coaching tailored to their given state of intoxication. Appropriate alarms and SOS messages may also be delivered if it is determined that this state of intoxication is severe or life threatening. Finally, the geofencing application and/or the pluggable testing device 16 may be activated if and when it is determined that the user has entered a vehicle, for example—the intervention application then providing appropriate coaching, alrets, SOS messages, etc.

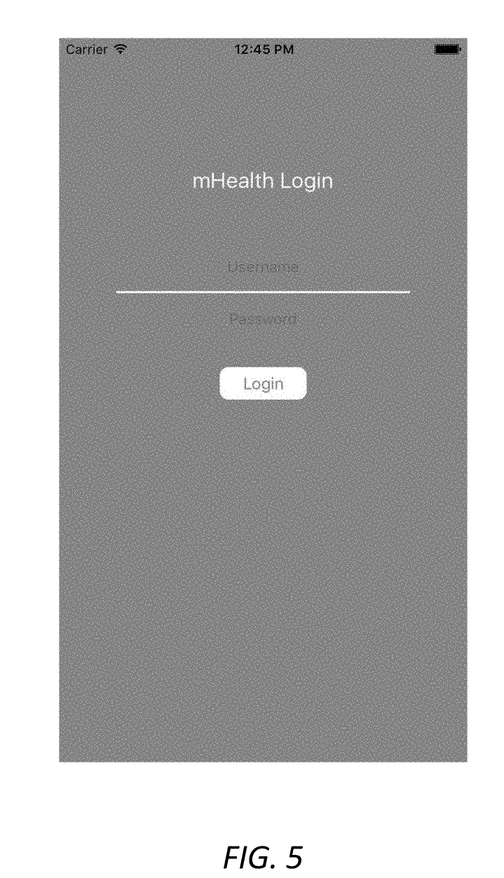

FIG. 5 is a mobile application screenshot of an exemplary authentication screen associated with the intervention system of the present invention. Appropriate credentials are provided to users during a registration process.

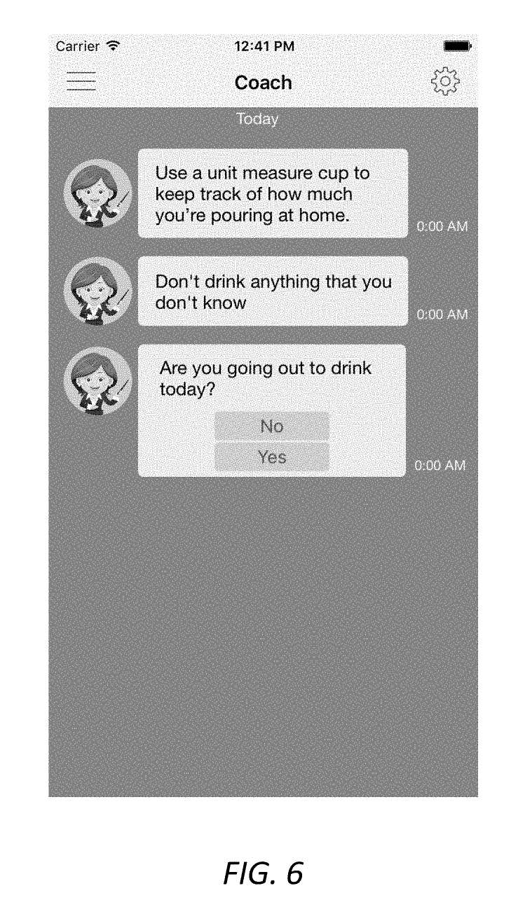

FIG. 6 is a mobile application screenshot of an exemplary coaching output screen associated with the intervention system of the present invention. Interaction with a user is provided through customized messages that are delivered to the user. These messages are preferably chained and depend upon user feedback, drinking history, and assessed progress, for example.

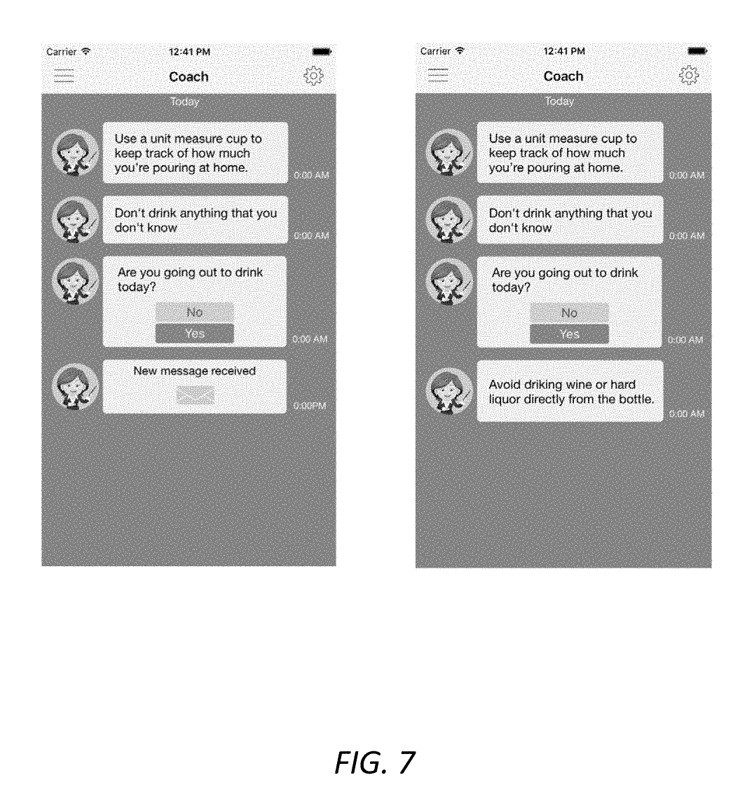

FIG. 7 is a mobile application dual screenshot of an exemplary coaching output screen associated with the intervention system of the present invention. Messages are blinded and displayed once a user clicks on them. This enables the provider to ensure that the user is actually reading the provided messages and progressing.

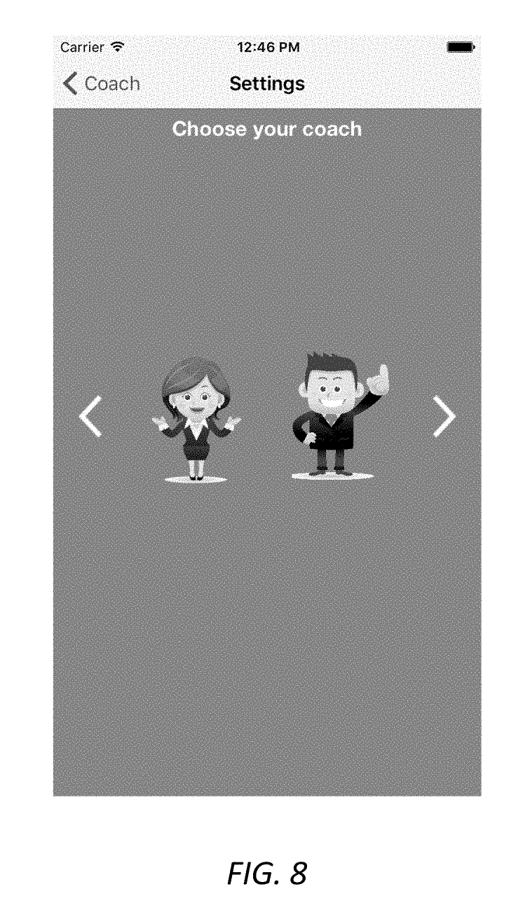

FIG. 8 is a mobile application screenshot of an exemplary coaching output screen associated with the intervention system of the present invention. A user is able to select a given virtual coach from a list of provided coaches, for example.

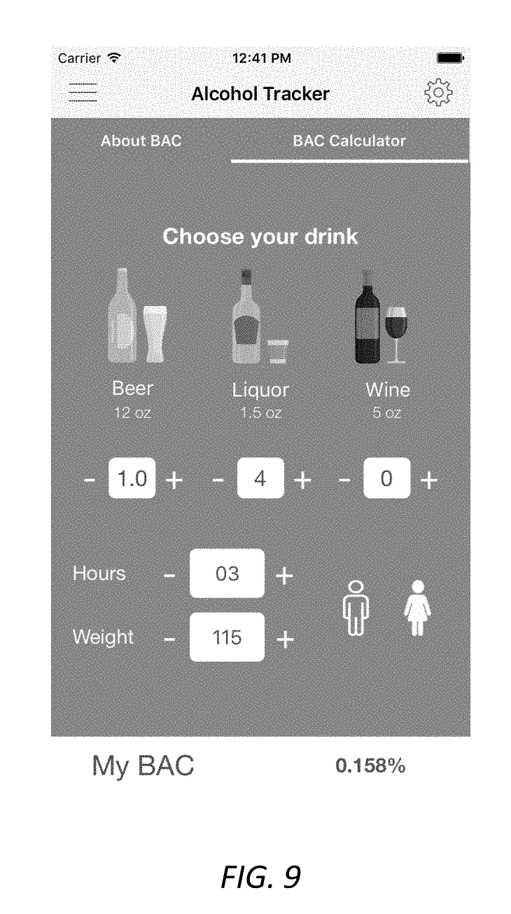

FIG. 9 is a mobile application screenshot of an exemplary BAC calculator screen associated with the intervention system of the present invention. Preferably, this BAC calculator is fully interactive and syncs with the other coaching functionalities.

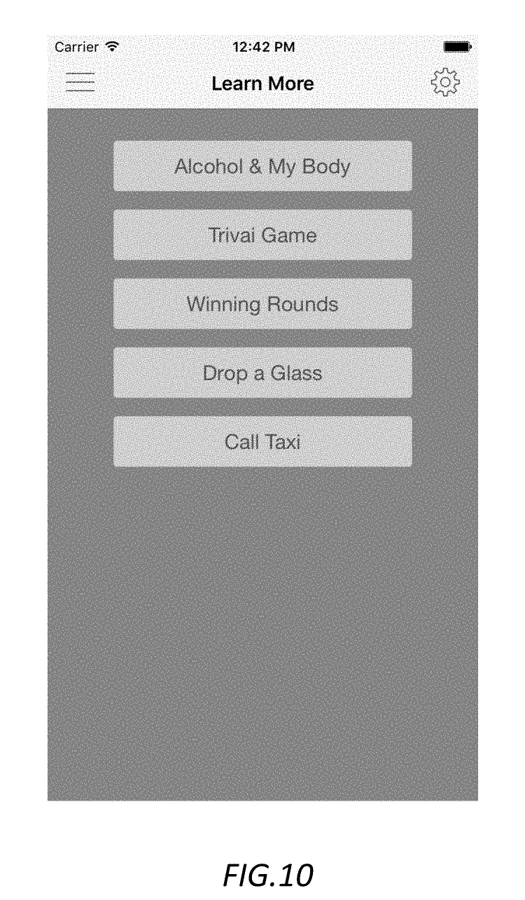

FIG. 10 is a mobile application screenshot of an exemplary learning resource screen associated with the intervention system of the present invention.

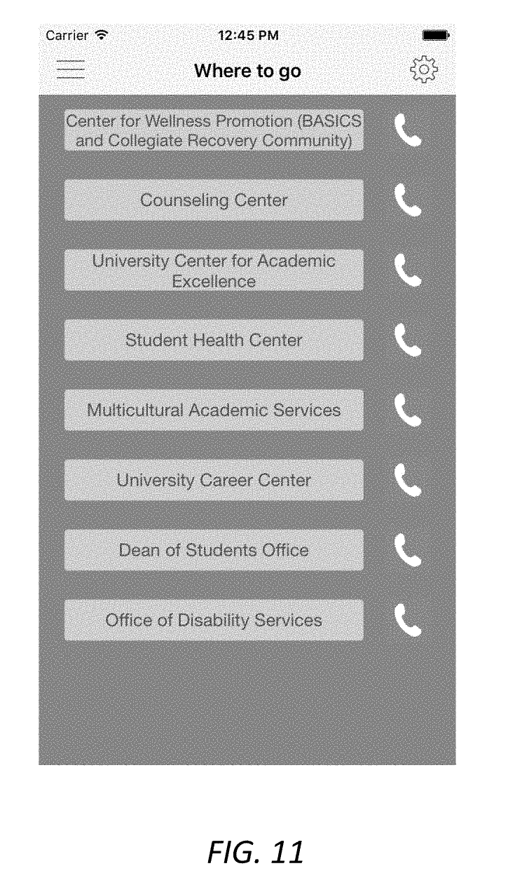

FIG. 11 is a mobile application screenshot of an exemplary contact list screen associated with the intervention system of the present invention. This provides the user with website urls and phone numbers for places that can provide the user with academic and health counseling assistance.

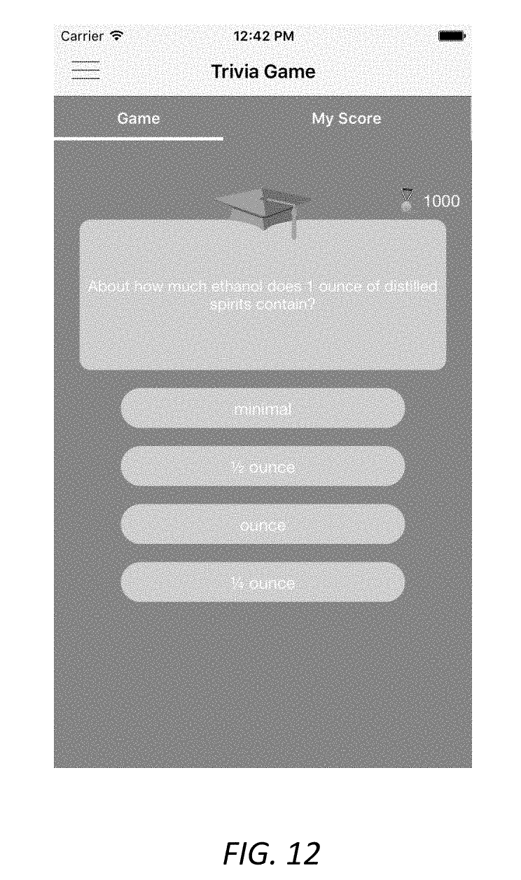

FIG. 12 is a mobile application screenshot of an exemplary trivia game screen associated with the intervention system of the present invention.

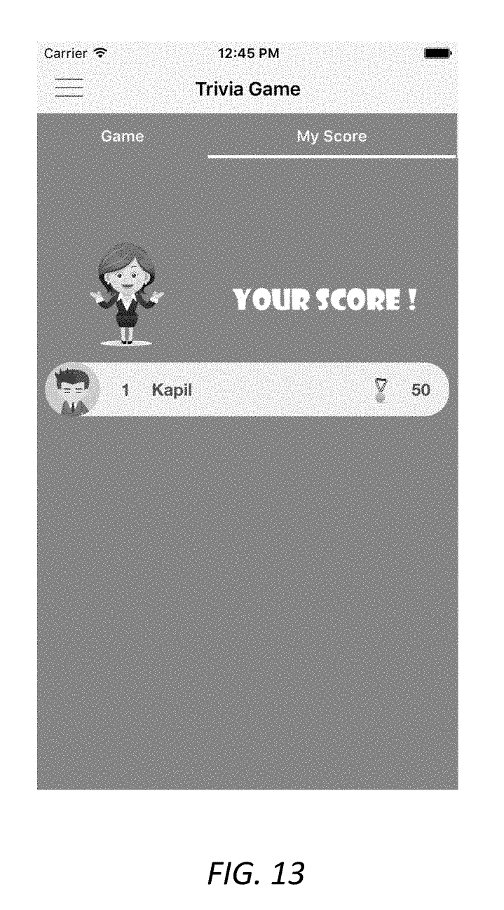

FIG. 13 is a mobile application screenshot of an exemplary trivia game scoreboard screen associated with the intervention system of the present invention. User identities are anonymized to ensure user privacy.

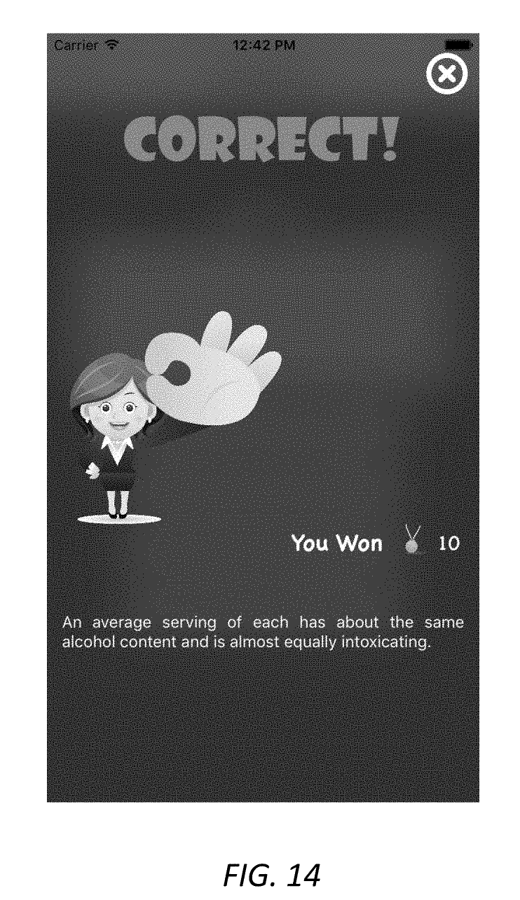

FIG. 14 is a mobile application screenshot of an exemplary trivia game feedback screen associated with the intervention system of the present invention.

Further, user authentication is provided using a username and password or through the provisioning of a barcode token that can be scanned by the mobile device camera to pass on the authentication token to the user. This authentication approach enables the user to login without the need to remember a username and password. It also allows the intervention councilor to be able to verify that the user is authenticated successfully.

The application does not expose the user identity and encrypts the user's information when stored in the database and in transit. Any user interactions or text input is analyzed to ensure that the submitted data does not expose the user's identity. This is done by building data mining text models that compute differential privacy metrics that validate that the user identity before and after making the post have the same exposure values.

Optionally, the mobile application provides several educational and awareness tools to the user. The user is provided with several trivia features that enhances the user's awareness of alcohol consumption in a fun and interesting way. The mobile application provides a tapping game that tests the user's attention to ensure that the user is not intoxicated and is able to focus. The user is asked to tap on several figures at specific times and time delays. The time delays are used to estimate the user's attention.

A student can virtually interact with the MI Coach engine or otherwise engage in games/activities in a virtual setting using a virtual reality device. A wearable biosensor device can be incorporated to provide biodata measures, including heart rate, respiratory rate, and skin temperature, for example. The information is used to identify a relaxation response to a virtual reality video, for example. A virtual game can be used to expose users to alcohol triggers and make drinking seem unpleasant. The virtual reality feature will reduce metabolic activity in the limbic system to train the brain to pay less attention to alcohol related stimuli, thus reducing consumption. The participants can play the sessions 10-15 minutes/day 4-5× week, for example. Participants can also exposed to different scenarios by the National Highway Transportation Safety Administration (NHTSA) that target millennials. Within the game, the user is exposed to alcohol situations then chooses whether to drive home; with the resulting consequences.

With the smartphone integrated breathalyzer, you breathe into the breathalyzer and the information is sent to your phone and can be found in the app to track participants daily drinking. It connects to your phone via Bluetooth. This will allow for photo/location-verified and time-stamped results with random testing. The information can also be integrated with the coach engine to redirect the intervention and messages. The BAC Track would have a normal sized breathalyzer which has police-grade Xtend Fuel Cell Sensors and one that is a keychain which uses a MicroCheck sensor.

The present invention, as a brief motivational intervention, is rooted in motivational interviewing and ecological momentary interventions—all approaches that can assist students in managing a myriad of issues that are known to impact college students' health, wellness, and safety. For instance, the present invention could be adapted to assist college students in quitting tobacco use, in achieving nutrition and exercise goals, or effectively managing stress—all issues that data show impact students' ability to succeed academically and co-curricularly. Use of a Fitbit wearable biosensor device to tract daily activities, can send reminders to move, and can track type of exercise and provides biodata measures including heart rate, respiratory rate, and skin temperature.

While the present invention has been designed to intervene specifically with college student populations, it is conceivable that this intervention could be adapted to address alcohol use (and possibly other issues such as PTSD, etc.) among military personnel. While the core concepts of being brief motivational and ecological momentary interventions, as well as overall architecture of the app would remain sound, the app could be appropriately adapted for military personnel (e.g., use of military jargon and acronyms, incorporating elements of military command structure and culture, etc.).

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following non-limiting claims.

What is claimed is:

1. An alcohol and drug intervention system, comprising:
a server component; and
a mobile application component wirelessly coupled to the server component;
wherein the server component and the mobile application component are collectively operable for, in an interactive manner, receiving input from a user and presenting the user with output based on the input received from the user, wherein the input received from the user comprises one or more of personal information, activity information, and activity related to alcohol and/or drug use, and wherein the output presented to the user comprises one or more of coaching information and counseling information.

2. The system of claim 1, wherein the server component comprises a processor executing one or more application programming interfaces and a database.

3. The system of claim 1, wherein the mobile application component comprises one or more of a mobile telephone, a personal digital assistant device, and a tablet device.

4. The system of claim 1, wherein the server component and the mobile application component are further collectively operable for presenting trivia game questions to the user and receiving trivia game answers from the user and adjusting one or more of the coaching and counseling information based on the trivia game answers received from the user.

5. The system of claim 1, wherein the server component and the mobile application component are further collectively operable for implementing a blood alcohol content calculator.

6. The system of claim 1, further comprising a geofencing component operable for determining a location of the user and tailoring requested inputs and presented outputs based thereon.

7. The system of claim 1, further comprising an alarm component operable for one or more of triggering and alarm and alerting a third party if the input received from the user indicates that user alcohol and/or drug use has exceeded a predetermined threshold.

8. The system of claim 1, further comprising a testing device coupled to the mobile application component and adjusting one or more of the coaching and counseling information based on results obtained from the testing device.

9. The system of claim 8, wherein the testing device comprises one or more of a breathalyzer, a saliva testing device, a blood testing device, a urine testing device, a facial expression analyzer.

10. The system of claim 1, wherein the server component and the mobile application component are further collectively operable for blinding an identity of the user such that the user is anonymous when interacting with the mobile application component.

11. An alcohol and drug intervention method, comprising:
providing a server component; and
providing a mobile application component wirelessly coupled to the server component;
wherein the server component and the mobile application component are collectively operable for, in an interactive manner, receiving input from a user and presenting the user with output based on the input received from the user, wherein the input received from the user comprises one or more of personal information, activity information, and activity related to alcohol and/or drug use, and wherein the output presented to the user comprises one or more of coaching information and counseling information.

12. The method of claim 11, wherein the server component comprises a processor executing one or more application programming interfaces and a database.

13. The method of claim 11, wherein the mobile application component comprises one or more of a mobile telephone, a personal digital assistant device, and a tablet device.

14. The method of claim 11, wherein the server component and the mobile application component are further collectively operable for presenting trivia game questions to the user and receiving trivia game answers from the user and adjusting one or more of the coaching and counseling information based on the trivia game answers received from the user.

15. The method of claim 11, wherein the server component and the mobile application component are further collectively operable for implementing a blood alcohol content calculator.

16. The method of claim 11, further comprising providing a geofencing component operable for determining a location of the user and tailoring requested inputs and presented outputs based thereon.

17. The method of claim 11, further comprising providing an alarm component operable for one or more of triggering and alarm and alerting a third party if the input received from the user indicates that user alcohol and/or drug use has exceeded a predetermined threshold.

18. The method of claim 11, further comprising providing a testing device coupled to the mobile application component and adjusting one or more of the coaching and counseling information based on results obtained from the testing device.

19. The method of claim 18, wherein the testing device comprises one or more of a breathalyzer, a saliva testing device, a blood testing device, a urine testing device, a facial expression analyzer.

20. The method of claim 11, wherein the server component and the mobile application component are further collectively operable for blinding an identity of the user such that the user is anonymous when interacting with the mobile application component.
